(19) United States
(12) Patent Application Publication
(10) Pub. No.: US 2016/0115470 A1
(43) Pub. Date: Apr. 28, 2016

(54) CELL FUSION DEVICE AND CELL FUSION METHOD

(71) Applicants: Senplus Inc., Gyeonggi-do (KR); Korea Advanced Institute Of Science And Technology, Daejeon (KR)

(72) Inventors: Young-Ho Cho, Daejeon (KR); Jong-Uk Bu, Gyeonggi-do (KR); Jiyoon Bu, Daejeon (KR)

(73) Assignees: Senplus Inc., Gyeonggi-do (KR); Korea Advanced Institute Of Science And Technology, Daejeon (KR)

(21) Appl. No.: 14/921,924

(22) Filed: Oct. 23, 2015

(30) Foreign Application Priority Data
Oct. 24, 2014 (KR) 10-2014-0145468

(51) Int. Cl. C12N 15/02; C12M 1/42; C12M 1/00

(52) U.S. Cl. CPC C12N 15/02; C12M 23/26; C12M 35/02

(57) ABSTRACT

A cell fusion device includes a chamber including a first input/output portion and a second input/output portion and providing a space through a fluid containing cells flows, at least one cell fusion structure provided in the chamber to form a fluidic channel through which the fluid flows and having a capturing portion adapted to capture the cell in the fluid and a fusion portion connected to the capturing portion and provide a space for fusing at least two cells trapped by the capturing portion and moved into the fusion portion in orders, a deformable membrane structure provided in the capturing portion of the cell fusion structure and configured to actuate to change a cross-sectional area of the fluidic channel of the capturing portion and to selectively capture the cell, and a membrane control portion configured to apply a pressure to the deformable membrane structure.

### PRIORITY STATEMENT

This application claims priority under 35 USC §119 to Korean Patent Application No. 10-2014-0145468, filed on Oct. 24, 2014 in the Korean Intellectual Property Office (KIPO), the contents of which are herein incorporated by reference in their entirety.

### FIELD OF THE DISCLOSURE

Example embodiments relate to a cell fusion device and a cell fusion method. More particularly, example embodiments relate to a device and method for cell pairing and fusion of cells in a fluid flowing through a micro fluidic channel.

### BACKGROUND OF THE DISCLOSURE

Generally, a cell pairing and fusion method using micro fluidics may include directing a solution having two types of cells to pass through a region to which an alternating current is applied or a region on which a material layer such as biotin-streptavidin is coated.

However, in these methods, only a limited proportion of cells' membranes is changed by electrical stimulus, and thus, throughput of cell pairing may be low. Also, cell pairing and fusion of undesired cells may frequently occur.

### SUMMARY OF THE DISCLOSURE

Example embodiments provide a cell fusion device with a high yield rate capable of performing a cell pairing and fusion of cells in a fluidic channel precisely.

Example embodiments provide a cell paring and fusion method using the cell fusion device.

According to example embodiments, a cell fusion device includes a chamber including a first input/output portion and a second input/output portion and providing a space through which a fluid containing cells flows, at least one cell fusion structure provided in the chamber to form a fluidic channel through which the fluid flows and having a capturing portion adapted to capture the cell in the fluid and a fusion portion connected to the capturing portion and provide a space for fusing at least two cells trapped by the capturing portion and moved into the fusion portion in orders, a deformable membrane structure provided in the capturing portion of the cell fusion structure and configured to actuate to change a cross-sectional area of the fluidic channel of the capturing portion and to selectively capture the cell, and a membrane control portion configured to apply a pressure to the deformable membrane structure.

In example embodiments, the cell fusion structure may include at least first and second channel patterns formed on an inner wall of the chamber to form the fluidic channel.

In example embodiments, the first and second channel patterns may be arranged to face each other to form the capturing portion and the fusion portion.

In example embodiments, an inlet of the cell fusion structure may have a first width, the capturing portion may have a second width greater than the first width, and the fusion portion may have a third width less than the second width.

In example embodiments, the deformable membrane structure may include a gate membrane portion which is deformed by the applied pressure to block the cell from entering the capturing portion. The gate membrane portion may be deformed by the applied pressure to close the inlet of the cell fusion structure.

In example embodiments, the membrane control portion may include a membrane pressurizing portion adapted to apply the pressure to the gate membrane portion.

In example embodiments, the membrane control portion may include a recess formed in an inner wall of the chamber to extend across the capturing portion of the cell fusion structure. The deformable membrane structure may include a deformable membrane to cover the recess.

In example embodiments, the membrane control portion may be connected to a pneumatic supply source and configured to deform the deformable membrane structure.

In example embodiments, the device may further include a pair of electrode patterns arranged in both sides of the cell fusion structure to apply an electrical signal to the cells placed in the fusion portion.

In example embodiments, the electrode patterns may be formed on an inner wall of the chamber.

In example embodiments, the device may further include an electrical signal generator connected to the electrode patterns to apply the electrical signal to the electrode patterns.

In example embodiments, a plurality of the cell fusion structures may be arranged in a first direction to form one capturing array.

In example embodiments, a plurality of the capturing arrays may be arranged in a second direction substantially perpendicular to the first direction.

According to example embodiments, in a cell fusion method, a cell fusion device is provided, the cell fusion device comprising a chamber including a first input/output portion and a second input/output portion, at least one cell fusion structure provided in the chamber to form a fluidic channel through which a fluid flows and having a capturing portion and a fusion portion connected to the capturing portion, and a deformable membrane structure provided in the capturing portion of the cell fusion structure. The deformable membrane structure is deformed to change a cross-sectional area of the fluidic channel of the capturing portion. A fluid containing cells is introduced into the chamber through the first input/output portion. At least two cells entering the fusion portion through the capturing portion are fused in the fusion portion.

In example embodiments, deforming the deformable membrane structure may include applying a pressure to the deformable membrane structure to block the cell entering the capturing portion.

In example embodiments, blocking the cell from entering through the capturing portion may include deforming the deformable membrane structure to close an inlet of the cell fusion structure.

In example embodiments, introducing the fluid containing cells into the chamber through the first input/output portion may include introducing a fluid containing a first cell and introducing a fluid containing a second cell.

In example embodiments, fusing the at least two cells may include applying an electrical signal to the cells placed in the fusion portion.

In example embodiments, the method may further include culturing the fused cell in the chamber.

In example embodiments, the method may further include analyzing characteristics of the fused cell in the chamber.

According to example embodiments, a plurality of cell fusion structures may function as cell pairing and fusion structures, together with a selectively deformable membrane structure. Thus, a cell fusion device may pair desired cells and fuse the paired cells, to thereby simply and precisely perform an electrical fusion or chemical fusion of the desired cells.

### BRIEF DESCRIPTION OF THE DRAWINGS

Example embodiments will be more clearly understood from the following detailed description taken in conjunction with the accompanying drawings. FIGS. 1 to 25 represent non-limiting, example embodiments as described herein.

FIG. 1 is an exploded perspective view illustrating a cell fusion device in accordance with example embodiments.

FIG. 2 is a plan view illustrating the cell fusion device in FIG. 1.

FIG. 3 is a plan view illustrating cell fusion structures in FIG. 1.

FIG. 4 is an enlarged view illustrating the cell fusion structure in FIG. 3.

FIG. 5 is a plan view illustrating membrane control lines in FIG. 1.

FIG. 6 is an enlarged view illustrating the A portion in FIG. 5.

FIG. 7 is a cross-sectional view taken along the A-A′ line in FIG. 2.

FIG. 8 is a plan view illustrating an electrical signal generator in FIG. 1.

FIG. 9 is a plan view illustrating the cell fusion structures and the membrane control line in FIG. 2.

FIGS. 10 and 11 are cross-sectional views taken along the B-B′ lines in FIG. 9.

FIGS. 12A to 12G are plan views illustrating a method of fusing cells in accordance with example embodiments.

FIGS. 13A to 13G are cross-sectional views respectively taken along the B-B lines in FIGS. 12A to 12G.

FIGS. 14A to 14D are plan views illustrating a cell fusion structure of a cell fusion device in accordance with example embodiments.

FIGS. 15A to 15D are plan views illustrating membrane control lines respectively corresponding to the cell fusion structures in FIGS. 14A to 14D.

FIG. 16 is a plan view illustrating a cell fusion structure in accordance with example embodiments.

FIGS. 17A to 17D are plan views illustrating a cell fusion structure in accordance with example embodiments.

FIG. 18A to 18C are plan views illustrating electrode patterns in accordance with example embodiments.

FIG. 19 is a plan view illustrating membrane control lines in accordance with example embodiments.

FIGS. 20A and 20B are plan views illustrating cell fusion structures in accordance with example embodiments.

FIGS. 21A and 21B are plan views illustrating membrane control lines respectively corresponding to the cell fusion structures in FIGS. 20A and 20B.

FIG. 22 is a plan view illustrating a first input/output portion in accordance with example embodiments.

FIG. 23 is a plan view illustrating a second input/output portion in accordance with example embodiments.

FIGS. 24A and 24B are plan views illustrating a chamber of a cell fusion device in accordance with example embodiments.

FIG. 25 is a plan view illustrating a first input/output portion of a cell fusion device in accordance with example embodiments.

### DETAILED DESCRIPTION OF THE ASPECTS OF THE DISCLOSURE

Various example embodiments will be described more fully hereinafter with reference to the accompanying drawings, in which some example embodiments are shown. The present inventive concept may, however, be embodied in many different forms and should not be construed as limited to the example embodiments set forth herein. Rather, these example embodiments are provided so that this description will be thorough and complete, and will fully convey the scope of the present inventive concept to those skilled in the art. In the drawings, the sizes and relative sizes of layers and regions may be exaggerated for clarity.

It will be understood that when an element or layer is referred to as being “on,” “connected to” or “coupled to” another element or layer, it can be directly on, connected or coupled to the other element or layer or intervening elements or layers may be present. In contrast, when an element is referred to as being “directly on,” “directly connected to” or “directly coupled to” another element or layer, there are no intervening elements or layers present. Like numerals refer to like elements throughout. As used herein, the term “and/or” includes any and all combinations of one or more of the associated listed items.

It will be understood that, although the terms first, second, third, fourth etc. may be used herein to describe various elements, components, regions, layers and/or sections, these elements, components, regions, layers and/or sections should not be limited by these terms. These terms are only used to distinguish one element, component, region, layer or section from another region, layer or section. Thus, a first element, component, region, layer or section discussed below could be termed a second element, component, region, layer or section without departing from the teachings of the present inventive concept.

Spatially relative terms, such as “beneath,” “below,” “lower,” “above,” “upper” and the like, may be used herein for ease of description to describe one element or feature's relationship to another element(s) or feature(s) as illustrated in the figures. It will be understood that the spatially relative terms are intended to encompass different orientations of the device in use or operation in addition to the orientation depicted in the figures. For example, if the device in the figures is turned over, elements described as “below” or “beneath” other elements or features would then be oriented “above” the other elements or features. Thus, the exemplary term “below” can encompass both an orientation of above and below. The device may be otherwise oriented (rotated 90 degrees or at other orientations) and the spatially relative descriptors used herein interpreted accordingly.

The terminology used herein is for the purpose of describing particular example embodiments only and is not intended to be limiting of the present inventive concept. As used herein, the singular forms “a,” “an” and “the” are intended to include the plural forms as well, unless the context clearly indicates otherwise. It will be further understood that the terms “comprises” and/or “comprising,” when used in this specification, specify the presence of stated features, integers, steps, operations, elements, and/or components, but do not preclude the presence or addition of one or more other features, integers, steps, operations, elements, components, and/or groups thereof.

Example embodiments are described herein with reference to cross-sectional illustrations that are schematic illustrations of idealized example embodiments (and intermediate structures). As such, variations from the shapes of the illustrations as a result, for example, of manufacturing techniques and/or tolerances, are to be expected. Thus, example embodiments should not be construed as limited to the particular shapes of regions illustrated herein but are to include deviations in shapes that result, for example, from manufacturing.

Unless otherwise defined, all terms (including technical and scientific terms) used herein have the same meaning as commonly understood by one of ordinary skill in the art to which this inventive concept belongs. It will be further understood that terms, such as those defined in commonly used dictionaries, should be interpreted as having a meaning that is consistent with their meaning in the context of the relevant art and will not be interpreted in an idealized or overly formal sense unless expressly so defined herein.

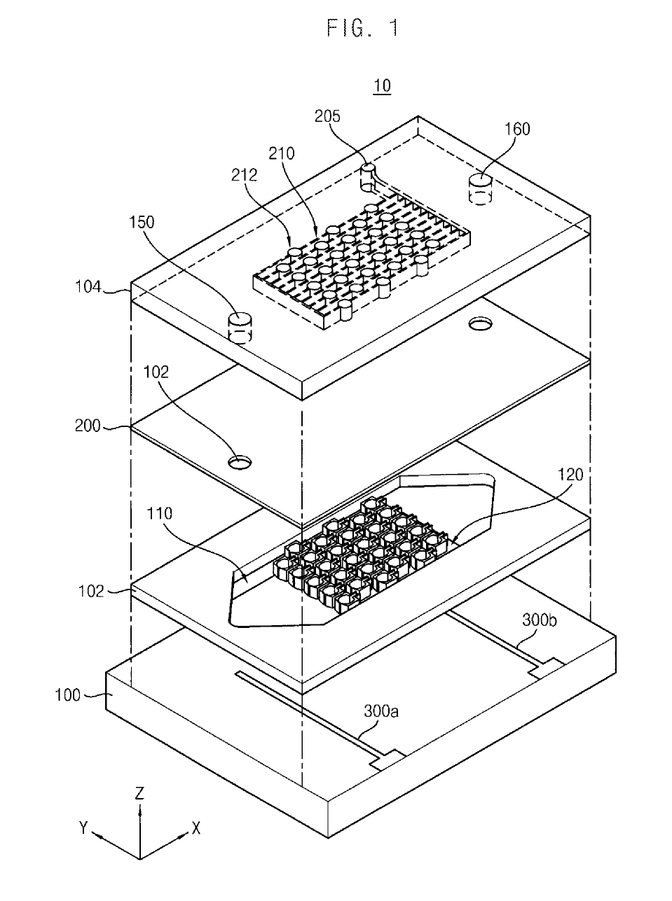

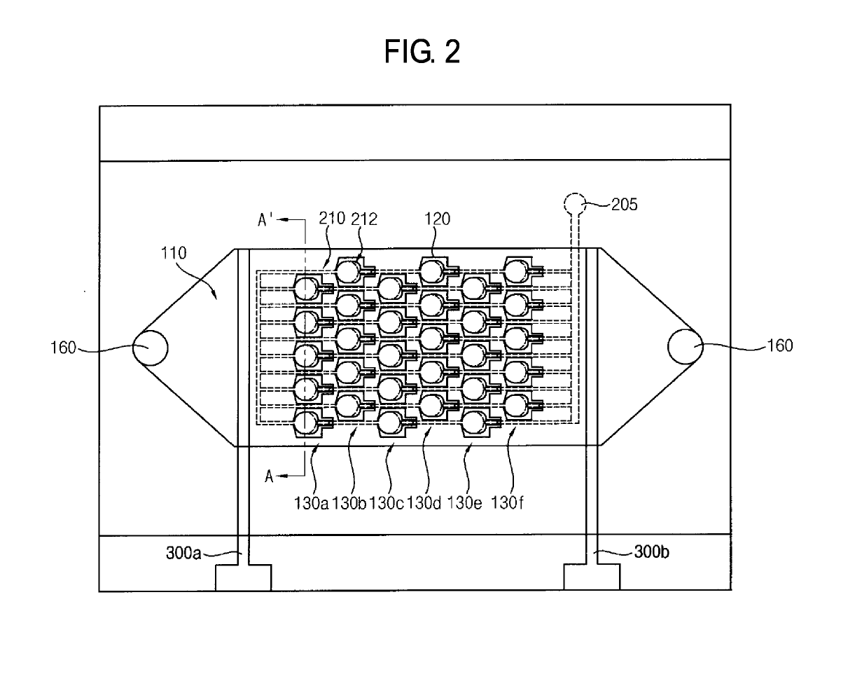

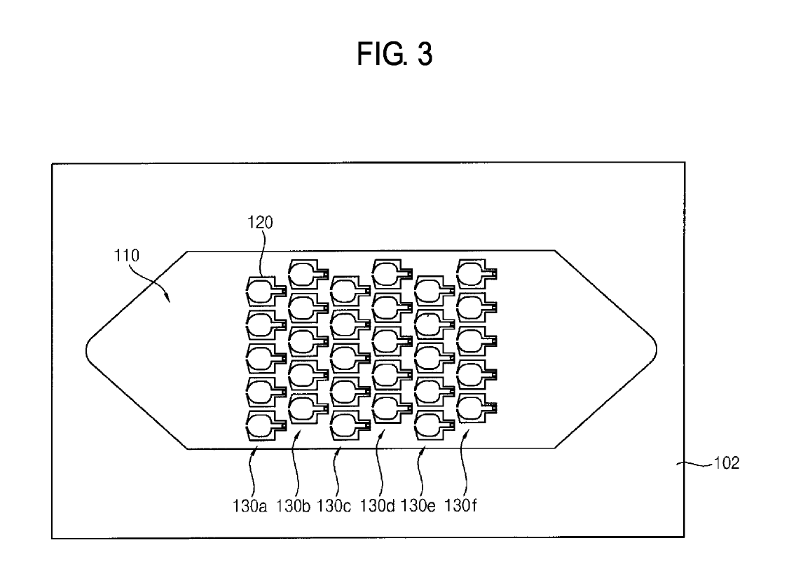

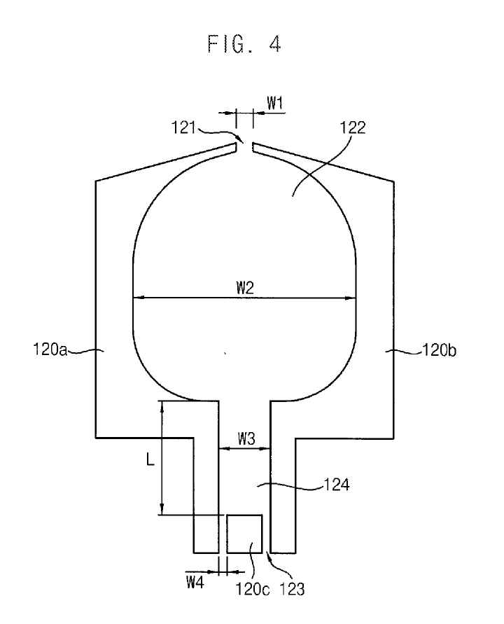

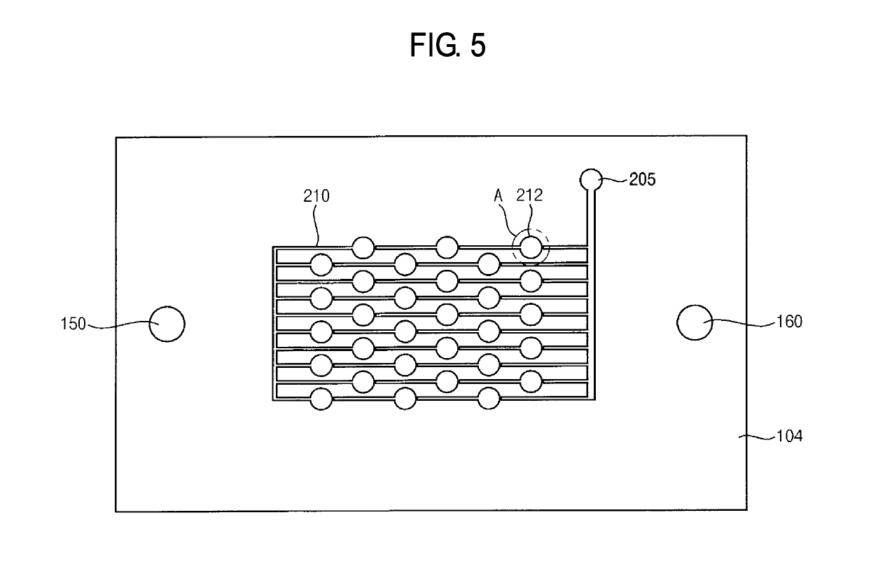

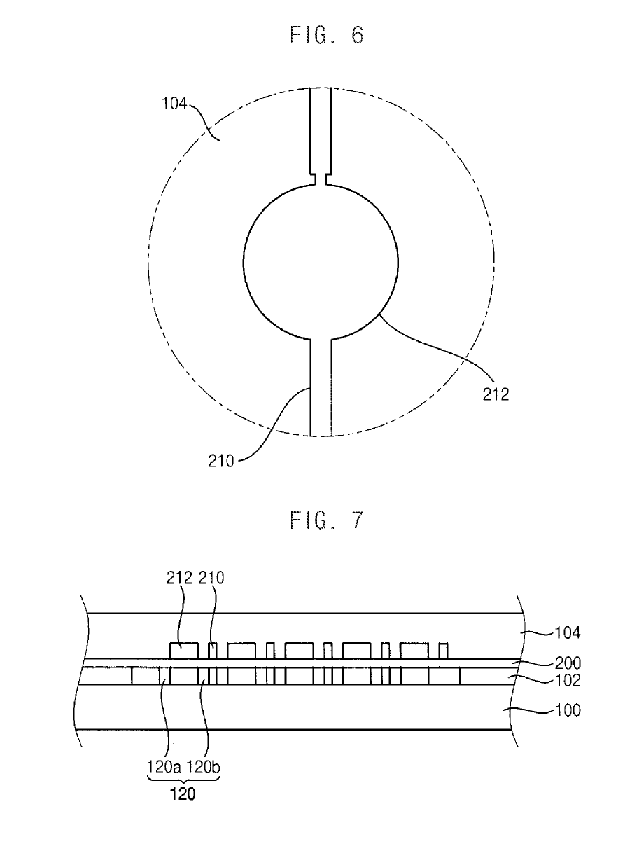

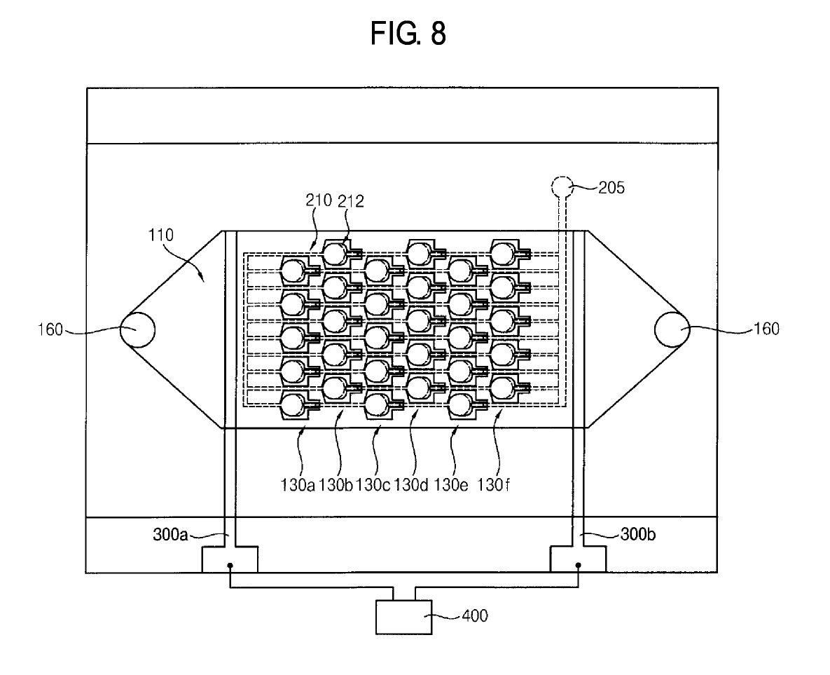

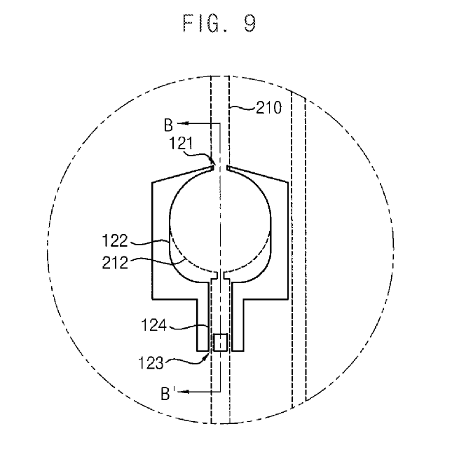

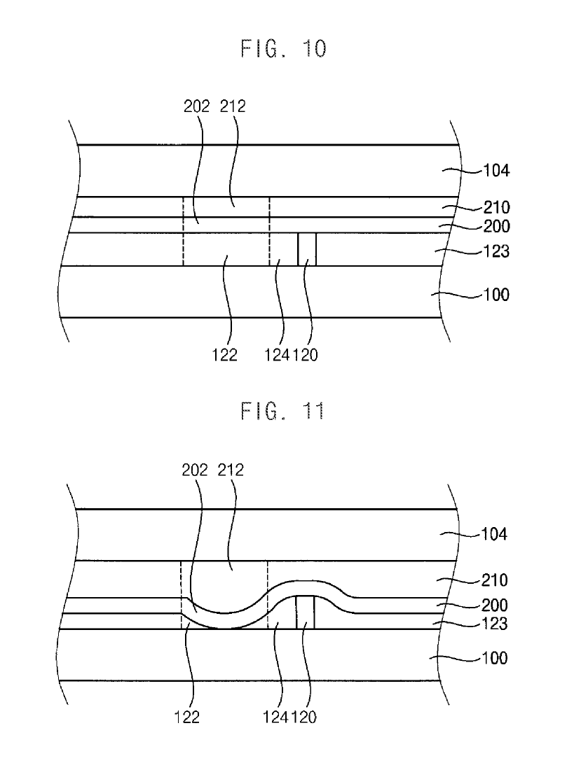

FIG. 1 is an exploded perspective view illustrating a cell fusion device in accordance with example embodiments. FIG. 2 is a plan view illustrating the cell fusion device in FIG. 2. FIG. 3 is a plan view illustrating cell fusion structures in FIG. 1. FIG. 4 is an enlarged view illustrating the cell fusion structure in FIG. 3. FIG. 5 is a plan view illustrating membrane control lines in FIG. 1. FIG. 6 is an enlarged view illustrating the A portion in FIG. 5. FIG. 7 is a cross-sectional view taken along the A-A′ line in FIG. 2. FIG. 8 is a plan view illustrating an electrical signal generator in FIG. 1. FIG. 9 is a plan view illustrating the cell fusion structures and the membrane control line in FIG. 2. FIGS. 10 and 11 are cross-sectional views taken along the B-B′ lines in FIG. 9.

Referring to FIGS. 1 to 11, a cell fusion device 10 may include a chamber 110, at least one capturing array 130a, 130b, 130c, 130d, 130e, 130f having a plurality of cell fusion structures 120 respectively. The cell fusion structures 120 may be configured to form a fluidic channel in the chamber 110 and selectively capture and fuse cells in a fluid flowing through the fluidic channel. Additionally, the cell fusion structures 120 may include a deformable membrane structure 202, and a membrane control line 210 may be configured as a membrane control portion to selectively apply a force (e.g., a pressure) to the deformable membrane structure. At least a pair of electrode patterns 300a and 300b may be disposed proximate opposing sides of the cell fusion structure 120 and may be configured to apply an electrical signal between the electrode patterns.

In example embodiments, the chamber 110 may include a first input/output portion 150 and a second input/output portion 160 disposed proximate opposing ends of the chamber 110 respectively. The chamber 110 may provide a space for fluid flow. The chamber 110 may have a polygonal shape when seen in plan view. For example, as shown in FIG. 1, the chamber 110 may have a hexagonal shape when seen in plan view. Although illustrated as having a hexagonal shape, one of ordinary skill in the art may appreciate the shape of the chamber 110 is not limited thereto and may have a circular shape, rectangular shape, a polygonal shape, and/or the like.

The fluid may enter the chamber 110 through the first input/output portion 150 and may exit the chamber 110 through the second input/output portion 160. In another aspect, a collecting fluid may flow into the chamber 110 through the second input/output portion 160 and flow out of the chamber 110 through the first input/output portion 150. For example, at least one fluid transfer element (not illustrated) may be connected to the first input/output portion 150 and/or the second input/output portion 160 and may be configured to supply the fluid into the chamber 110 and/or remove the fluid from the chamber 110. Additionally or alternatively, the fluid may be transferred through the chamber 110 by rotating or tilting the device 10. In this case, the rotational speed, the rotational acceleration and/or the rotational direction of the chamber 110, and/or the inclination, orientation, and/or the like of the device 10 may be controlled to adjust the flow rate of the fluid.

In some aspects, the fluid may be a solution containing biochemical particles such as, for example, different types of cells. Examples of the biochemical particles may include fibroblasts, embryonic stem cells, myeloma cells, and/or the like. Further, the fluid may include a polymer, which is not a biochemical, configured to chemically fuse the cells trapped in the cell fusion structure. For example, the fluid may include polyethylene glycol (PEG), which is widely used in chemical cell fusion processes.

The chamber 110, the cell fusion structures 120, the deformable membrane structure, the membrane control line 210 and/or the electrode pattern 300a, 300b may be formed by a semiconductor manufacturing processes such as, for example photolithography, ion lithography, electron lithography, and/or the like. The chamber 110 may be formed using polymeric materials and/or inorganic materials. Examples of the polymeric material may be PDMS (polydimethylsiloxane), PMMA (polymethylmethacrlyate), SU-8, and/or the like. Examples of the inorganic material may be glass, quartz, silicon, and/or the like. The electrode pattern may be formed using a metal material. Examples of the metal material may be gold, silver, platinum, copper, aluminum, and/or the like.

As illustrated in FIGS. 1 and 2, the cell fusion device 10 may include a first substrate 100, a second substrate 102, a deformable membrane 200, and a third substrate 104 that are assembled in a stacked relationship with respect to one another. For example, the first substrate 100, the second substrate 102, the deformable membrane 200, and the third substrate 104 may be arranged with respect to one another such that the first substrate 100 substantially abuts the second substrate 102, the second substrate 102 substantially abuts the first substrate 100 and the deformable membrane 200, the deformable membrane 200 substantially abuts the second substrate 102 and the third substrate 104, and the third substrate 104 substantially abuts the deformable membrane 200.

In some aspects, the second substrate 102 may be formed on the first substrate 100 and may partially define the chamber 110 and first to sixth capturing arrays 130a, 130b, 130c, 130d, 130e and 130f, which may each include a plurality of the cell fusion structures 120 arranged within the chamber 110. The first to sixth capturing arrays 130a, 130b, 130c, 130d, 130e and 130f may be arranged sequentially in a first direction (i.e., direction along X-axis) from the first input/output portion 150 to the second input/output portion 160 in the chamber 110. Alternatively, an opening may be formed in a single substrate so as to define the chamber, and the cell fusion structures may be formed in the opening of the single substrate. Further, heights of the cell fusion structures 120 may be substantially the same as or less than a height of the second substrate 102.

According to some aspects, the deformable membrane 200 may be stacked on the second substrate 102, and the third substrate 104 may be stacked on the second substrate 102. According to another aspect, the deformable membrane 200 may be stacked on the second substrate 102, and the third substrate 104 may be stacked on the deformable membrane 200. That is, the deformable membrane 200 may be disposed interposed between the second substrate 102 and the third substrate 104. The deformable membrane 200 may operably engage the second substrate 102 so as to enclose the chamber 110, cover the cell fusion structures 120, and define at least one fluidic channel. The fluidic channel may be defined by an upper surface of the first substrate 100, a lower surface of the deformable membrane 200, and surfaces of the cell fusion structure 120. The third substrate 104 may define the at least one membrane control line 210 configured to deform a portion of the deformable membrane (i.e., the deformable membrane structure), which forms a portion of the fluidic channel.

In particular, the third substrate 104 may define a recess that opens towards the first and second substrates 100 and 102 when the first substrate 100, the second substrate 102, the deformable membrane 200, and the third substrate 104 are arranged to form the cell fusion device 10. In some embodiments, the recess may form the membrane control line. The recess may extend along the first direction or along a second direction (direction along Y-axis) that is perpendicular to the first direction. Additionally, the recess may form the membrane control line that extends across the cell fusion structure 120. The deformable membrane 200, when operably engaged with the third substrate, may cover the recess so as to provide the deformable membrane structure, which constitutes one wall of the fluidic channel. In some aspects, the upper surface of the first substrate 100 may constitute a bottom wall of the chamber 110 and/or the fluidic channel, and the lower surface of the third substrate 104 may constitute an upper wall of the chamber 110.

A plurality of the recesses may be formed in the lower surface of the third chamber 104 to form a plurality of the membrane control lines 210. For example, membrane control lines may be arranged spaced apart from each other along the second direction. The membrane control lines may extend along the first direction across at least one cell fusion structure 120. The membrane control lines may be connected to a common pneumatic supply source 205. Thus, the deformable membrane structure may be arranged in the cell fusion structure 120. Additionally, the deformable membrane structure 202 may be deformed when a force (i.e., pressure) is applied to the deformable membrane structure by the membrane control line 210.

The deformable membrane 200 may define a first hole 250 that is in fluid communication with the first input/output portion 150. Additionally or alternatively, the deformable membrane 200 may define a second hole 252 that is in fluid communication with the second input/output portion 160. Accordingly, the fluid may be introduced to the chamber 110 via the first hole 250 and the first input/output portion 150, and the fluid may be removed from the chamber 110 via the second input/output portion 160 and the second hole 252.

When a fluid flows in a first flow direction from the first input/output portion 150 to the second input/output portion 160 in the chamber 110, the fluid may pass sequentially the first, second, third, fourth, fifth and sixth capturing arrays 130a, 130b, 130c, 130d, 130e and 130f. In some embodiments, the first flow direction may provide for capturing particles in the fluid.

When a fluid flows in a second flow direction from the second input/output portion 160 to the first input/output portion 150 in the chamber 110, the fluid may pass sequentially the sixth, fifth, fourth, third, second and first capturing arrays 130f, 130e, 130d, 130c, 130b and 130a, and may provide for collecting the captured particles.

The first capturing array 130a may include a plurality of the cell fusion structures 120 arranged to be spaced from each other in the second direction (Y direction) perpendicular to the first direction. Similarly, the second to sixth capturing arrays 130b, 130c, 130d, 130e and 130f may include a plurality of the cell fusion structures 120 arranged substantially the same as or similar to the cell fusion structures 120 of the first capturing array 130a.

As illustrated in FIG. 4, the cell fusion structure 120 may include a pair of first and second channel patterns 120a and 120b disposed within the chamber 110 so as to form a fluidic channel through which a fluid may flow. In some embodiments, the pair of first and second channel patterns 120a and 120b may be disposed on an inner wall that partially defines the chamber 110. The first and second channel patterns 120a and 120b may be shaped symmetrically with respect to each other. The first and second channel patterns 120a and 120b may be arranged to face each other to form a capturing portion 122 and a fusion portion 124. The fusion portion 124 may be connected to the capturing portion 122 and may define a space in which the particles entering through the capturing portion 122 are received.

Front end portions of the first and second channel patterns 120a and 120b may form an inlet 121 through which the fluid flows into the capturing portion 122. Rear end portions of the first and second channel patterns 120a and 120b, together with a third channel pattern 120c interposed between the first and second channel patterns 120a and 120b, may form an outlet 123 through which the fluid flows out of the fusion portion 124.

The inlet 121 of the cell fusion structure 120 may have a first size (e.g., width W1) such that a deformable particle in the fluid can enter the capturing portion 122 while being deformed under a hydraulic pressure. The outlet 123 of the cell fusion structure 120 may have a second size (e.g., width W4) such that the deformable particle in the fluid cannot escape the fusion portion through the outlet even though the particle is deformed under a hydraulic pressure. The capturing portion 122 may have a second width W2 greater than the first width W1 and the fusion portion 124 may have a third width W3 less than the second width W2. The capturing portion 122 may have a first length and the fusion portion 124 may have a second length L, which may be the same as or different from the first length of the capturing portion 122. The lengths of the capturing portion 122 and the fusion portion 124 may be determined with consideration towards the number and sizes of the particles to be captured and fused.

When a fluid flows in the first flow direction in the chamber 110, the fluid may pass through the fluidic channel of the cell fusion structure 120. As mentioned herein, when the capturing portion 122 is opened, the particle in the fluid may enter the cell fusion structure 120 through the capturing portion 122 and be captured in the fusion portion 124.

FIG. 10 represents an original position of the deformable membrane structure when an air pressure is not applied to the membrane control line in FIG. 9. FIG. 11 represents a deformed position of the deformable membrane structure when an air pressure is applied to the membrane control line in FIG. 9.

As illustrated in FIGS. 2, and 9 to 11, the membrane control line 210 may extend across the capturing portion 122 of the cell fusion structure 120. The membrane control line 210 may include a membrane pressurizing portion 212 configured to expand the corresponding portion of the deformable membrane 200 in the capturing portion 122. For example, the membrane pressurizing portion 212 may have a circular shape when seen in plan view corresponding to the shape of the capturing portion 122 of the cell fusion structure 120.

Accordingly, a gate membrane portion 202, that is, the deformable membrane structure may be disposed in the capturing portion 122 of the cell fusion structure 120 to be controlled by the membrane pressurizing portion 212. Thus, the deformable membrane structure may include the gate membrane portion 202 disposed in the capturing portion 122. The membrane control lines 210 may be connected to the pneumatic supply source 205 to control the gate membrane portion 202.

For example, when a cell to be captured in a fluid has a diameter of about 15 μm to 25 μm, the inlet 121 of the cell fusion structure 120 may have a width W1 of about 7 μm to 10 μm and the outlet 123 of the cell fusion structure 120 may have a width W4 of about 1 μm to 3 μm. Dimensions of the cell fusion structure, including the widths of the inlet 121 and the outlet 123 may be determined with consideration of deformation characteristics of the cell. The channel pattern of the cell fusion structure 120 may have a height of about 20 μm to 30 μm. The membrane pressurizing portion 121 may have a diameter of about 130 μm to 160 μm. When an air pressure of about 80 kPa is applied to the membrane control line 210, the maximum deformed displacement (i.e., height along Z direction) of the deformable membrane structure may range from about 60 μm to 100 μm.

As illustrated in FIGS. 10 and 11, when a force (e.g., air pressure) is applied to the membrane control line 210, the membrane pressurizing portion 212 may deform the gate membrane portion 202. The gate membrane portion 202 may be deformed by the applied pressure to close the capturing portion 122 such that the particle in the fluid may be blocked from entering through the capturing portion 122. The gate membrane portion 202 may be arranged adjacent to the inlet 121 of the cell fusion structure 120, and the gate membrane portion 202 may be deformed to close at least a portion of the inlet 121 to prevent the particle from entering the capturing portion 122. When the inlet 121 of the cell fusion structure 120 is partially closed, the particle in the fluid may be deformed by a hydraulic pressure to be temporarily captured in the inlet 121.

The diameter of the gate membrane portion may be determined according to the diameter of the membrane pressurizing portion. In some aspects, the gate membrane portion may have a width (diameter) capable of capturing only one cell.

When the gate membrane portion 202 is deformed, because the fusion portion 124 has a relatively smaller width W3 than the width W2 of the capturing portion, the deformed length of the deformable membrane structure into the fusion portion 124 may be relatively smaller. Accordingly, even when the gate membrane portion 202 is deformed, the particle disposed in the fusion portion 124 may not be affected and/or deformed by the deformable membrane structure.

When the air pressure is discharged from the membrane control line 210, the gate membrane portion 202 may be returned elastically to its original position. When the gate membrane portion 220 returns to its original position, the inlet 121 of the cell fusion structure 120 may be opened completely and a particle may be transferred through the capturing portion 122 and may travel to the fusion portion 124.

As illustrated in FIG. 8, the cell fusion device 10 may further include an electrical signal generator 400 configured to provide an electrical signal to the at least a pair of the electrode patterns 300a and 300b disposed on opposing sides of the cell fusion structure 120. The first electrode pattern 300a and the second electrode pattern 300b may extend along the second direction. The capturing array may be disposed between the first and second electrode patterns 300a and 300b. The first and second electrode patterns 300a and 300b may be disposed proximate opposing sides of the chamber 110 respectively. The electrical signal generator 400 may be connected to connection terminals of the first and second electrode patterns 300a and 300b and may be configured to provide an electrical signal (e.g., alternating voltage) to the electrode patterns. The different type of cells sequentially placed in the fusion portion 124 of the cell fusion structure 120 may be fused by applying the electrical signal between the first and second electrode patterns 300a and 300b.

As mentioned above, the gate membrane portion 202 may be arranged in the capturing portion 122 of the cell fusion structure 120, and the gate membrane portion 202 may be pressurized selectively by the corresponding membrane pressurizing portion 212 to capture only one cell in the fluid. The gate membrane portion may be selectively deformed, to control the number and types of the particles and/or cells received in the fusion portion 124 from the capturing portion 122. The different types of the particles and/or cells received in the fusion portion 124 may be fused by a chemical solution, by an electrical signal, and/or by any other suitable means. Further, the fused cells may be response-analyzed or cell-cultured in the fusion portion 124.

Accordingly, a plurality of the cell fusion structures 120, together with the deformable membrane structure, may function as cell pairing and fusion structures. Thus, the cell fusion device 10 may form a plurality of paired cells, fuse the paired cells, and simply and precisely perform an electrical fusion or chemical fusion of desired cells.

Hereinafter, a method for cell pairing and fusion using the cell fusion device in FIG. 1 will be explained in detail.

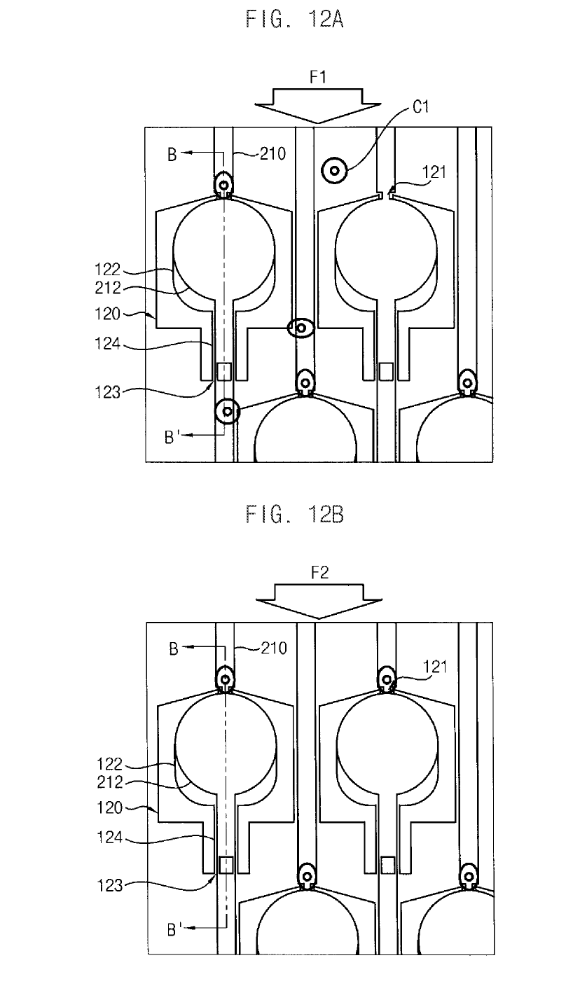

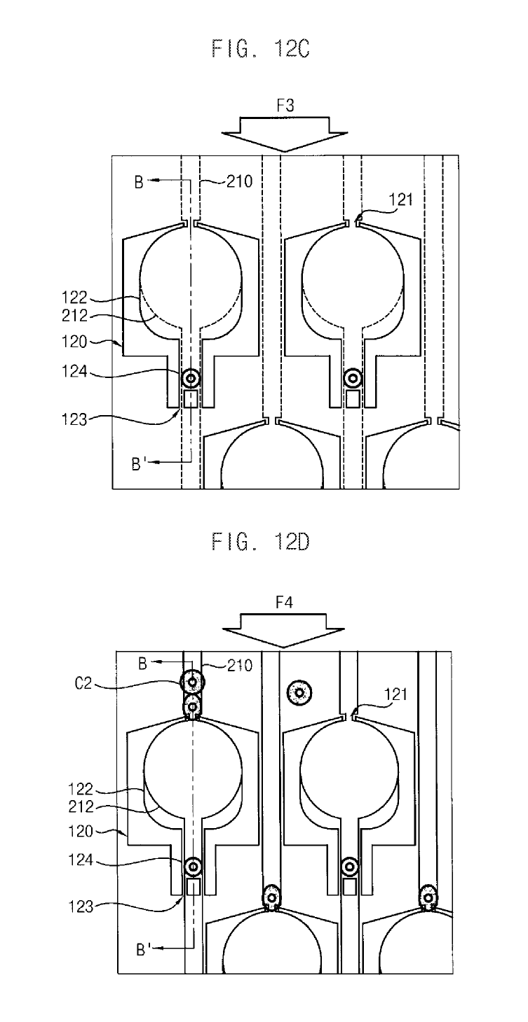

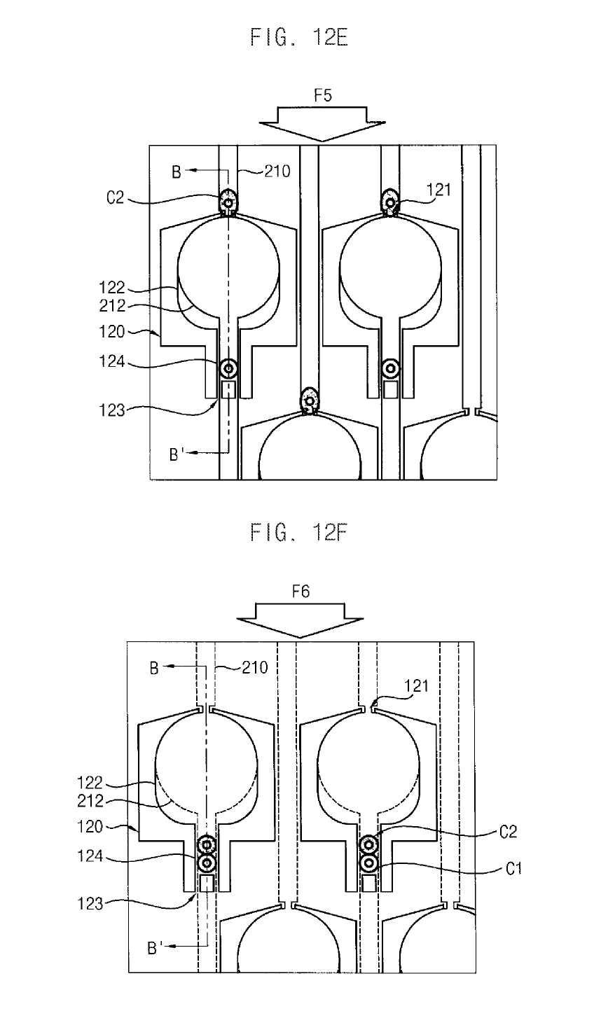

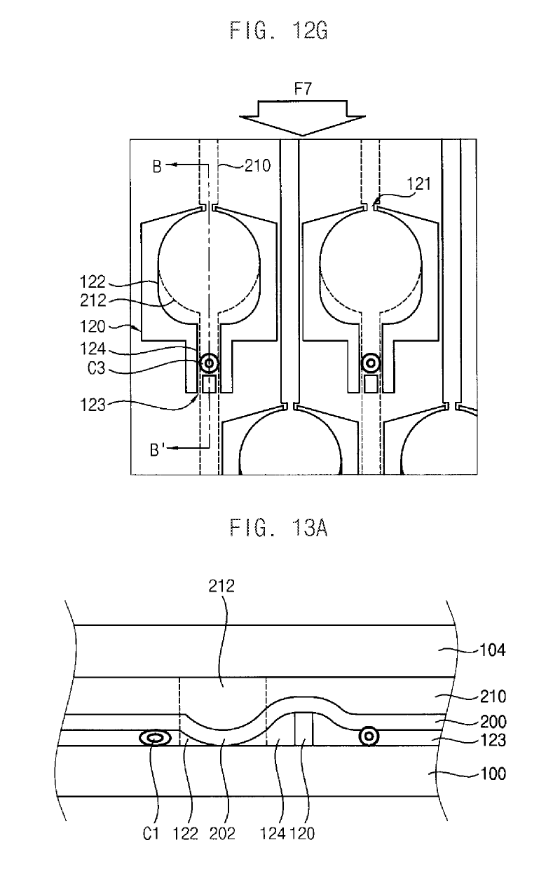

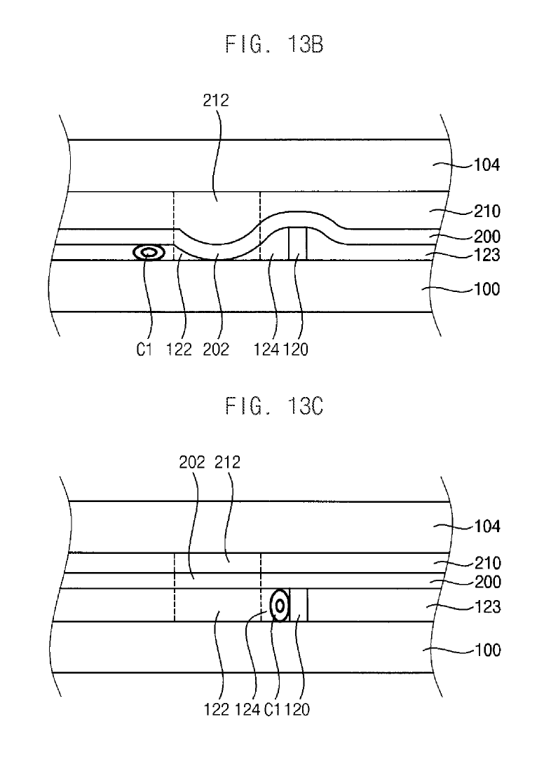

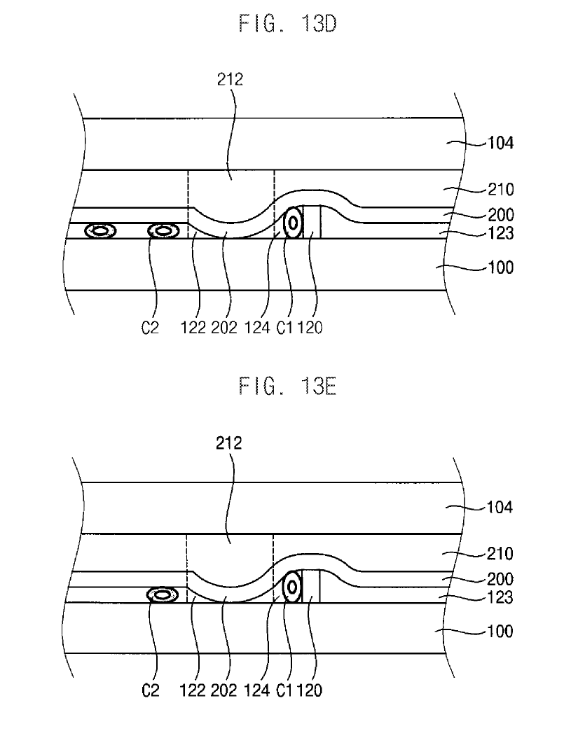

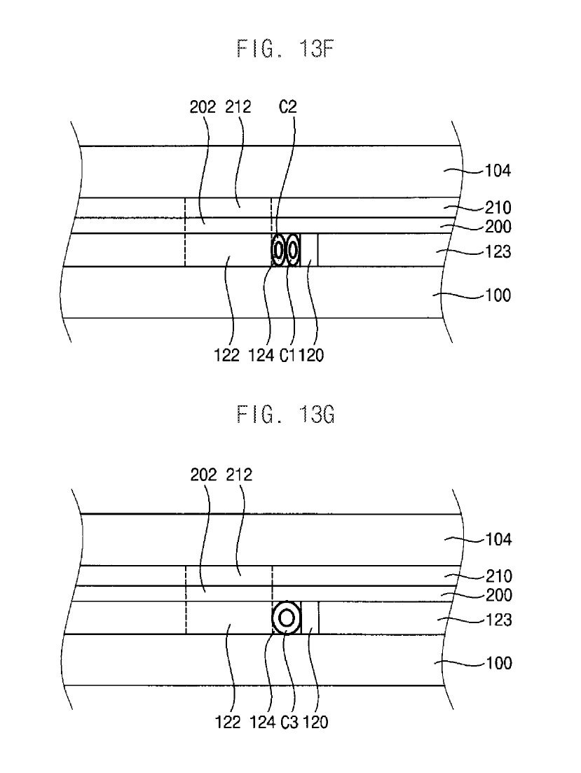

FIGS. 12A to 12G are plan views illustrating a method of fusing cells in accordance with example embodiments. FIGS. 13A to 13G are cross-sectional views respectively taken along the B-B lines in FIGS. 12A to 12G.

Referring to FIGS. 12A and 13A, first, a force (e.g., air pressure) may be applied via the membrane control line 210 to deform the gate membrane portion 202. Then, a first fluid F1 that includes first cells C1 may be introduced into the chamber 110 through the first input/output portion 150.

Thus, the gate membrane portion 202 may be deformed by the membrane pressurizing portion 212 of the membrane control line 210 to block the first cell C1 from entering the capturing portion 122 of the cell fusion structure 120. The inlet 121 of the cell fusion structure 120 adjacent to the capturing portion 122 may be partially closed, and the first cell C1 may be temporarily disposed in the inlet 121 while being deformed by a hydraulic pressure.

Referring to FIGS. 12B and 13B, a second fluid F2 without particles may be introduced into the chamber 110 through the first input/output portion 150 and drained from the chamber 110 through the second input/output portion 160. Thus, uncaptured first cells C1 may be discharged from the chamber 110 by the flow of the second fluid F2.

Referring to FIGS. 12C and 13C, the air pressure may be discharged from the membrane control line 210 to return the gate membrane portion 202 to its original position. Then, a third fluid F3 without particles may be introduced into the chamber 110 through the first input/output portion 150.

Thus, the first cell C1 captured in the inlet 121 may be transferred into the fusion portion 124 by passing through the capturing portion 122. Accordingly, one first cell C1 may be trapped in the fusion portion 124 of the cell fusion structure 120.

Referring to FIGS. 12D and 13D, a force (e.g., air pressure) may be applied via the membrane control line 210 to deform the gate membrane portion 202. Then, a fourth fluid F4 containing second cells C2 may be introduced into the chamber 110 through the first input/output portion 150.

Thus, the gate membrane portion 202 may be deformed by the membrane pressurizing portion 212 of the membrane control line 210 to block the second cell C2 from entering the capturing portion 122 of the cell fusion structure 120. In here, the inlet 121 of the cell fusion structure 120 adjacent to the capturing portion 122 may be partially closed, and the second cell C2 may be temporarily captured in the inlet 121 while being deformed by a hydraulic pressure.

Referring to FIGS. 12E and 13E, a fifth fluid F5 without particles may be introduced into the chamber 110 through the first input/output portion 150 and drained from the chamber 110 through the second input/output portion 160 so as to remove any uncaptured second cells C2 from the chamber 110.

Referring to FIGS. 12F and 13F, the air pressure may be discharged from the membrane control line 210 to return the gate membrane portion 202 to its original position. Then, a sixth fluid F6 without particles may be introduced into the chamber 110 through the first input/output portion 150. Thus, the second cell C2 captured in the inlet 121 may be transferred into the fusion portion 124 by passing through the capturing portion 122. Accordingly, one second cell C2 may be trapped in the fusion portion 124 of the cell fusion structure 120.

Referring to FIGS. 12G and 13G, the first cell C1 and the second cell C2 disposed sequentially within the fusion portion 124 of the cell fusion structure 120 may be fused together.

For example, a seventh fluid F7 containing a polymer for chemical cell fusion, such as PEG may be introduced into chamber 110 through the first input/output portion 150 to form a fused cell C3. An electrical signal for electrical cell fusion may be applied to the first and second electrode patterns 300a and 300b to fuse the first cell C1 and the second cell C2 to form a fused cell C3.

Thus, two types of the cells may be precisely and simply captured in the fusion portion 124 of the cell fusion structure 120 so as to be chemically or electrically cell-fused. Further, the fused cell may be response-analyzed or cell-cultured in the fusion portion 124.

In example embodiments, the cell fusion device 10 may further include a chemical or biological material layer coated on an inner wall of the chamber 110 or the deformable membrane structure. The material layer may be formed on the inner wall of the chamber to increase or decrease an adhesive strength with the particle and/or cell. Alternatively, the material layer may be formed by performing a surface treatment on any of the surfaces that define the chamber 110. For example, the material layer such as collagen may be coated on the first substrate 100.

Further, the cell fusion device 10 may include an additional structure fixed on the gate membrane portion or the sidewall of the chamber to assist in capturing a particle. The cell fusion device 10 may further include electrodes disposed on opposing sides of the cell fusion structure 120 or the capturing array to count the particles.

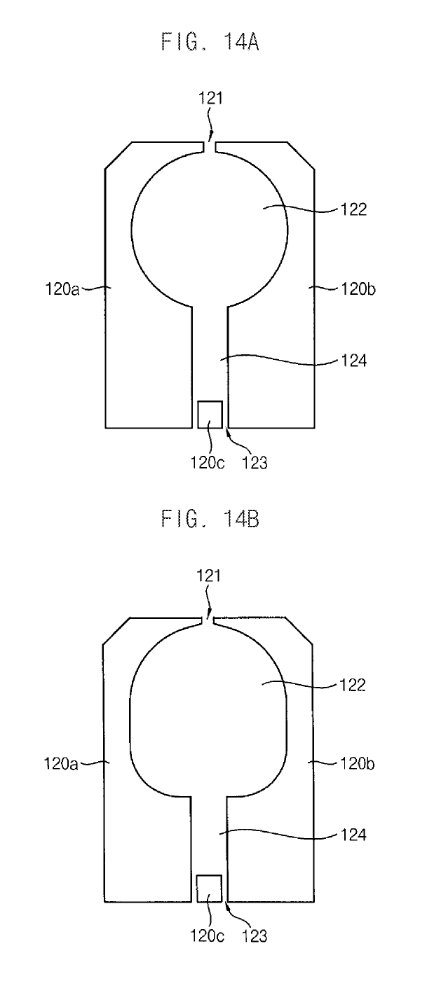

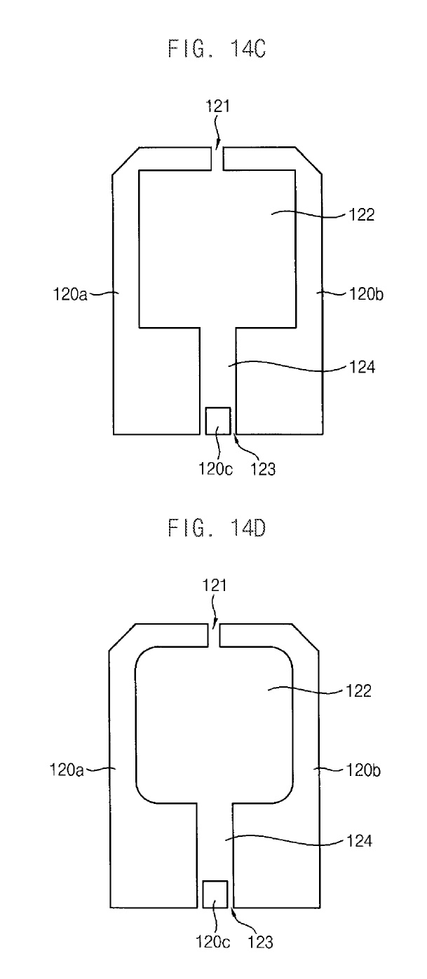

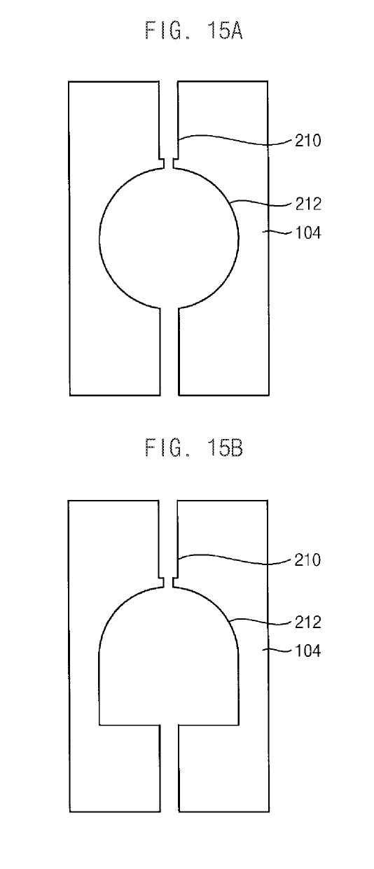

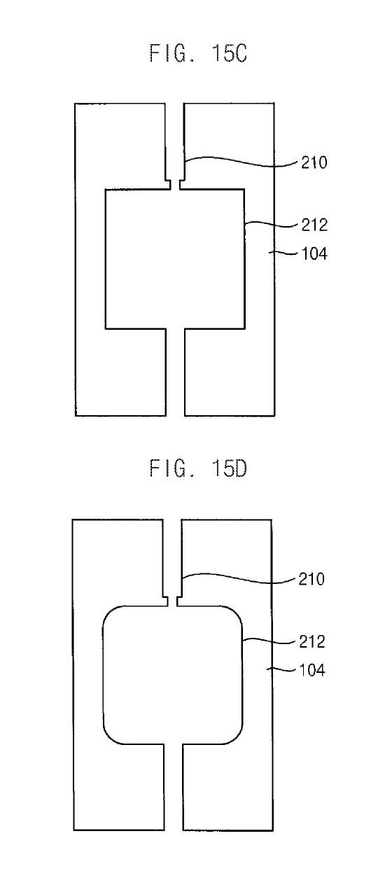

FIGS. 14A to 14D are plan views illustrating a cell fusion structure of a cell fusion device in accordance with example embodiments. FIGS. 15A to 15D are plan views illustrating membrane control lines respectively corresponding to the cell fusion structures in FIGS. 14A to 14D.

Referring to FIGS. 14A to 15D, a pair of first and second channel patterns 120a and 120b of a cell fusion structure 120 may be symmetric to each other. A distance between the first and second channel patterns 120a and 120b may be changed along an extending direction thereof to form an inlet and an outlet of the cell fusion structure 120. Widths of the first and second channel patterns 120a and 120b may be changed along the extending direction.

A capturing portion 122 formed by the first and second channel patterns 120a and 120b may have a circular shape, a polygonal shape or a combination thereof when seen in plan view. A membrane pressurizing portion of a membrane control line 210 may have shape corresponding to the capturing portion 122.

FIG. 16 is a plan view illustrating a cell fusion structure in accordance with example embodiments.

Referring to FIG. 16, a fusion portion 123 of a cell fusion structure 120 may have a length L. The number of cells fused in the fusion portion 124 may be determined by the length L of the fusion portion 123. For example, a first cell C1 and a second cell C2 to be fused together and a third cell C3 and a fourth cell C4 to be fused together may be received in the fusion portion 124. A non-biochemical particle P may be captured to be arranged between the first and second cells C1 and C2 and the third and fourth cells C3 and C4.

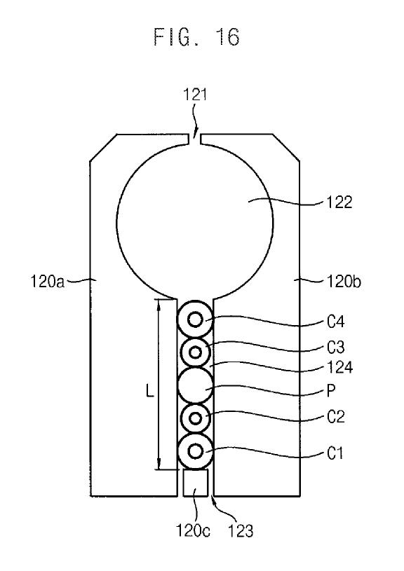

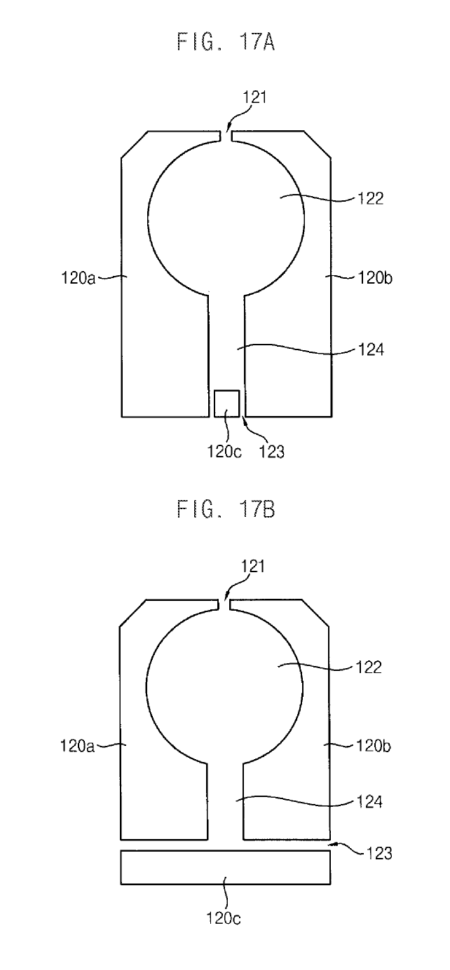

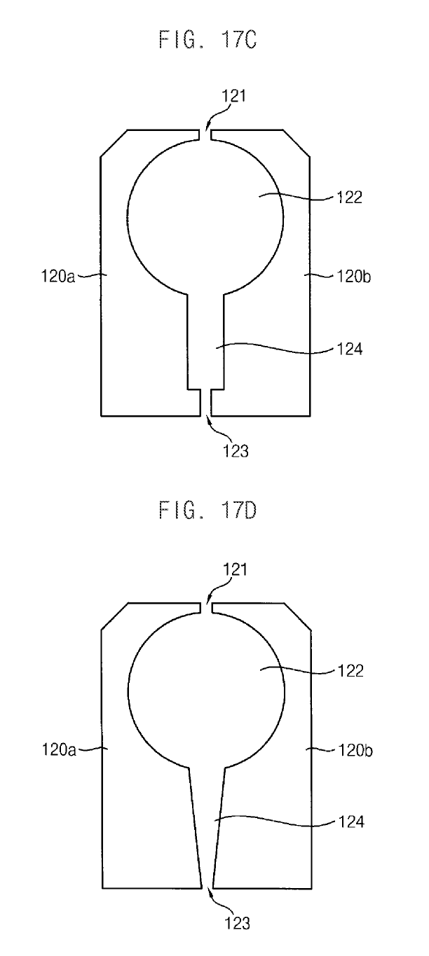

FIGS. 17A to 17D are plan views illustrating a cell fusion structure in accordance with example embodiments.

Referring to FIGS. 17A and 17B, a third channel pattern 120c may be arranged adjacent to rear end portions of first and second channel patterns 120a and 120b to form an outlet 123 through which a fluid is drained. The number of the outlets 123 and an outflow direction may be determined by a shape and arrangement of the third channel pattern 120c.

Referring to FIGS. 17C and 17D, a pair of first and second channel patterns 120a and 120b of a cell fusion structure 120 may be symmetric to each other. A distance between the first and second channel patterns 120a and 120b may be changed along an extending direction thereof to form an inlet 121 and an outlet 123 of the cell fusion structure 120. Widths of the first and second channel patterns 120a and 120b may be changed along the extending direction.

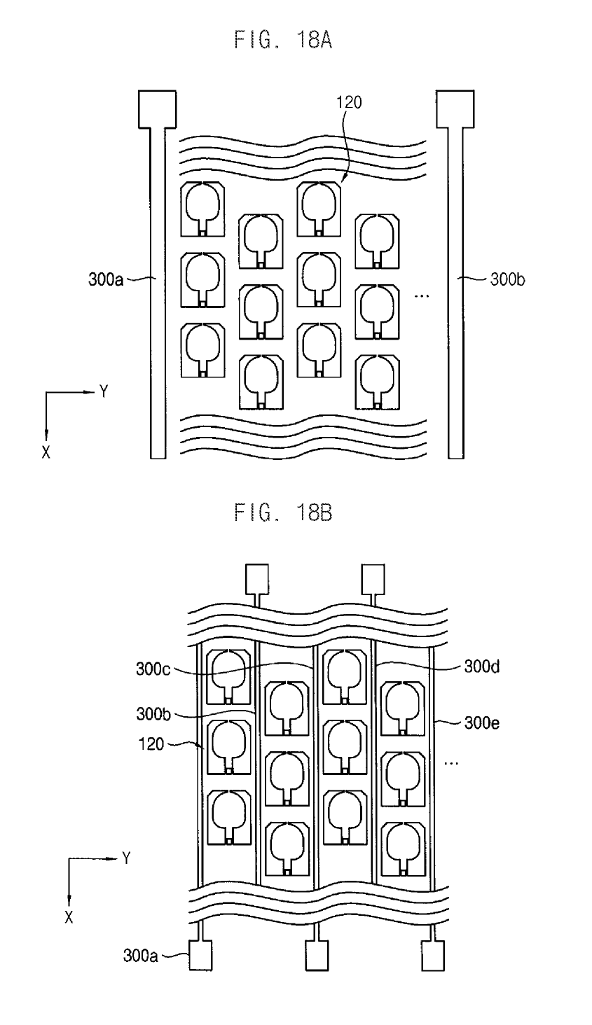

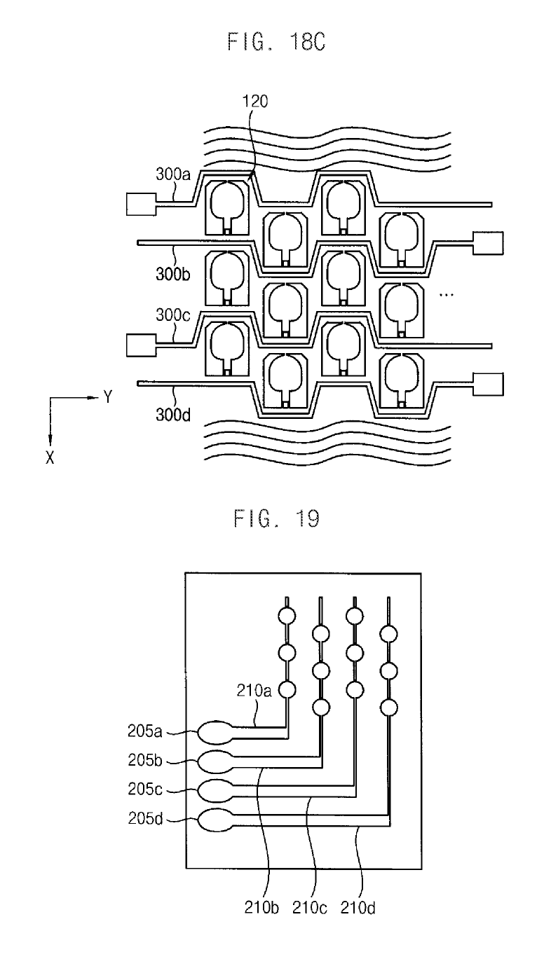

FIG. 18A to 18C are plan views illustrating electrode patterns in accordance with example embodiments.

Referring to FIG. 18A, a first electrode pattern 300a and a second electrode pattern 300b may extend in a first direction (X direction) respectively. The first electrode pattern 300a and the second electrode pattern 300b may be arranged to be spaced apart from each other in a second direction (Y direction). Capturing arrays may be interposed between the first and second electrode patterns 300a and 300b. The first and second electrode patterns 300a and 300b may be arranged in both sides of a chamber respectively.

Referring to FIG. 18B, a first electrode pattern 300a, a second electrode pattern 300b, a third electrode pattern 300c, a fourth electrode pattern 300d and a fifth electrode pattern 300e may extend in a first direction (X direction) respectively and arranged to be spaced apart from each other in a second direction (Y direction). A first column of cell fusion structures 120 may be interposed between the first and second electrode patterns 300a and 300b. A second column of the cell fusion structures 120 may be interposed between the second and third electrode patterns 300b and 300c. A third column of the cell fusion structures 120 may be interposed between the third and fourth electrode patterns 300c and 300d. A fourth column of the cell fusion structures 120 may be interposed between the fourth and fifth electrode patterns 300d and 300e. Accordingly, a cell fusion process may be selectively performed in any one column of the first to fourth columns of the cell fusion structures 120.

Referring to FIG. 18C, a first electrode pattern 300a, a second electrode pattern 300b, a third electrode pattern 300c and a fourth electrode pattern 300d and a fifth electrode pattern 300e may extend in a second direction (Y direction) respectively and arranged to be spaced apart from each other in a first direction (X direction). For example, a first row of cell fusion structures 120 may be arranged in a zigzag manner along the second direction (Y direction). Similarly, a second row of the cell fusion structures 120 and a third row of the cell fusion structures 120 may be arranged in a zigzag manner along the second direction. The first row of the cell fusion structures 120 may be interposed between the first and second electrode patterns 300a and 300b. The second row of the cell fusion structures 120 may be interposed between the second and third electrode patterns 300b and 300c. The third row of the cell fusion structures 120 may be interposed between the third and fourth electrode patterns 300c and 300d. Accordingly, a cell fusion process may be selectively performed in any one row of the first to third rows of the cell fusion structures 120.

FIG. 19 is a plan view illustrating membrane control lines in accordance with example embodiments.

Referring to FIG. 19, a plurality of membrane control lines may be connected to individual pneumatic supply sources to operate independently from one another. A first membrane control line 210a may be connected to a first pneumatic supply source 205a and extend in a first direction (X direction) to cross a first column of cell fusion structures in a chamber. A second membrane control line 210b may be connected to a second pneumatic supply source 205b and extend in the first direction to cross a second column of the cell fusion structures. A third membrane control line 210c may be connected to a third pneumatic supply source 205c and extend in the first direction to cross a third column of the cell fusion structures. A fourth membrane control line 210d may be connected to a fourth pneumatic supply source 205d and extend in the first direction to cross a fourth column of the cell fusion structures.

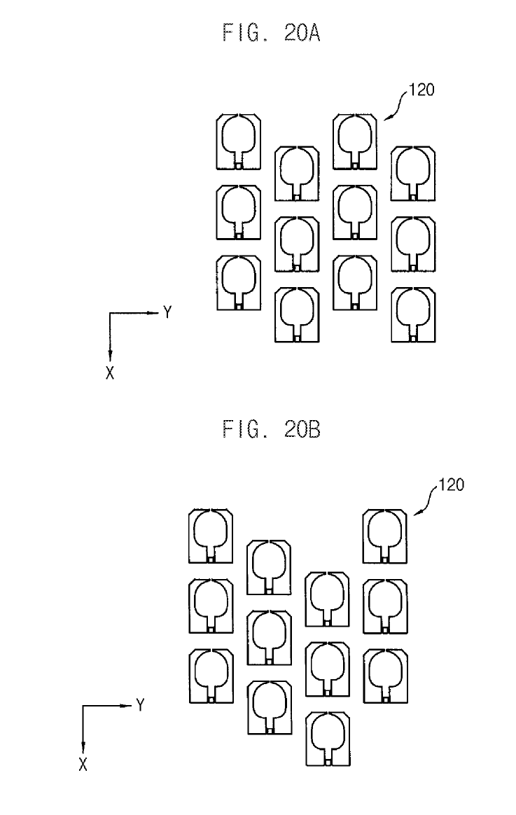

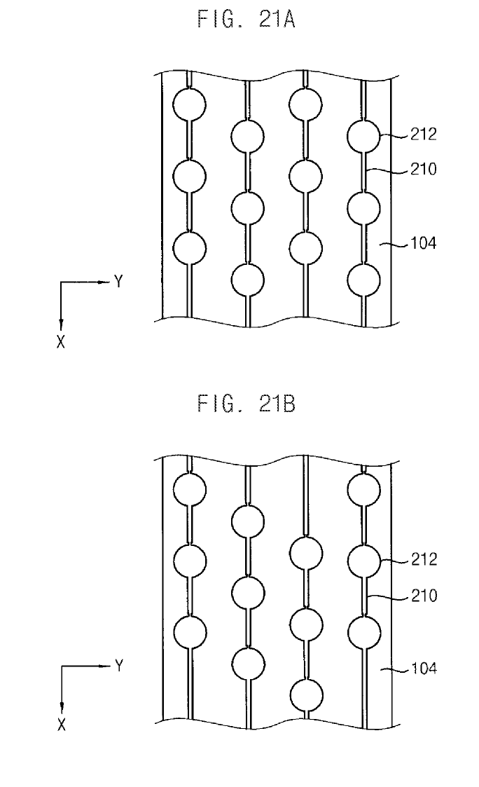

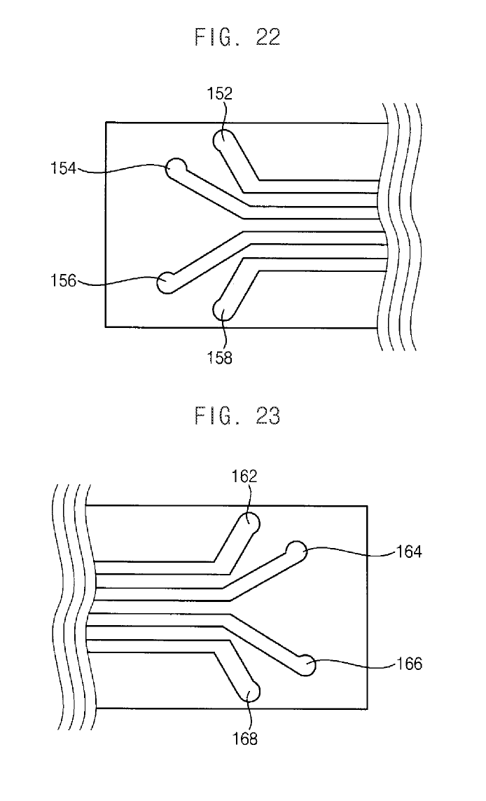

FIGS. 20A and 20B are plan views illustrating cell fusion structures in accordance with example embodiments. FIGS. 21A and 21B are plan views illustrating membrane control lines respectively corresponding to the cell fusion structures in FIGS. 20A and 20B.

Referring to FIGS. 20A and 21A, cell fusion structures 120 may be arranged in various patterns in a chamber. The cell fusion structures 120 arranged in a second direction (Y direction) may form one capturing array. The cell fusion structures 10 may be arranged in a zigzag manner along the second direction. A plurality of the capturing arrays may be arranged in a first direction (X direction). Membrane control lines 210 may extend to cross the cell fusion structures respectively. One membrane control line may extend to cross one column of the cell fusion structures. The membrane control line 210 may include a membrane pressurizing portion 212 corresponding to each of the cell fusion structures 120. A distance between the cell fusion structures 120, a size of the membrane pressurizing portion 212, etc. may be determined in consideration of an arrangement of the cell fusion structures, a size of a particle, etc.

FIG. 22 is a plan view illustrating a first input/output portion in accordance with example embodiments.

Referring to FIG. 22, a first input/output portion may include a plurality of inflow/outflow portions 152, 154, 156, 158. A fluid containing particles may be introduced into a chamber through the inflow/outflow portions. Alternatively, fluid containing different types of particles may be introduced into the chamber through the inflow/outflow portions sequentially or simultaneously. Some of the inflow/outflow portions may be used to provide a pressure for fluid flow or supply a fluid for collecting particles or for cleaning the chamber.

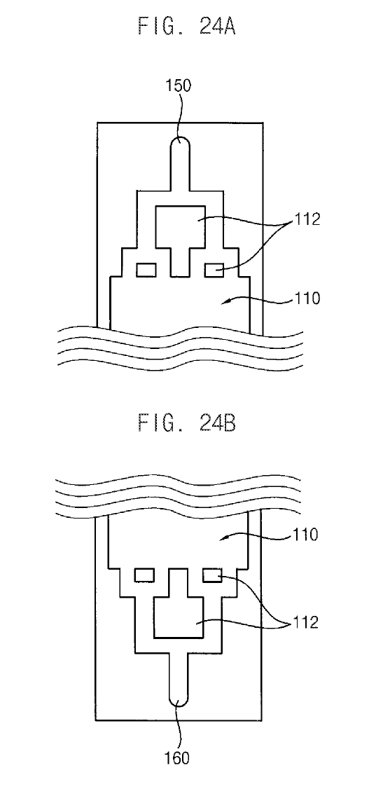

FIG. 23 is a plan view illustrating a second input/output portion in accordance with example embodiments.

Referring to FIG. 23, a second input/output portion may include a plurality of inflow/outflow portions 162, 164, 166, 168. A fluid containing particles may be drained from a chamber through the inflow/outflow portions. Same or different types of particles may be collected through the inflow/outflow portions. Some of the inflow/outflow portions may be used to provide a pressure for fluid flow or supply a fluid for collecting particles or for cleaning the chamber.

FIGS. 24A and 24B are plan views illustrating a chamber of a cell fusion device in accordance with example embodiments.

Referring to FIGS. 24A and 24B, a cell fusion device may further include a guiding structure 112 arranged in a chamber 110. The guiding structure 112 may guide a fluid to run smoothly through the chamber 110. The guiding structure may control a mixture of fluids or a distribution of fluid flow.

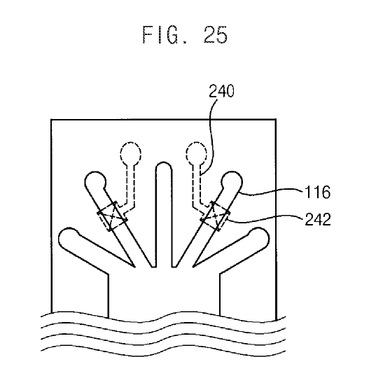

FIG. 25 is a plan view illustrating a first input/output portion of a cell fusion device in accordance with example embodiments. The cell fusion device is substantially the same as or similar to the cell fusion device described with reference to FIG. 1, except a valve assembly for controlling inflow path. Thus, the same or like reference numerals will be used to refer to as the same or like elements and any repetitive explanation concerning the above elements will be omitted.

Referring to FIG. 25, a cell fusion device may include a deformable valve structure 242 provided in an inflow path 116 to open or close the inflow path 116 and a valve control line 240 adapted to apply a pressure to the deformable valve structure 242. The valve control line 240 may include a recess which is formed in an inner wall of a chamber, for example, a surface of a second substrate to extend in a direction. The deformable valve structure 242 may cover the recess to form an air pressure line and constitute a sidewall of the inflow path 116. Accordingly, when the valve control line 240 is filled up with an air pressure, the deformable valve structure 242 may be deformed by the air pressure to close the inflow path 116. When the air pressure is discharged from the valve control line 240, the deformable valve structure 242 may be returned elastically to its original position to open the inflow path 116.

Although it is not illustrated in the figures, the deformable valve structure and the valve control line may be provided in an outflow path of a second input/output portion. Further, an external fluid supply line may be connected to the inflow path of the first input/output portion, and an external valve assembly may be installed in the external fluid supply line.

The foregoing is illustrative of example embodiments and is not to be construed as limiting thereof. Although a few example embodiments have been described, those skilled in the art will readily appreciate that many modifications are possible in the example embodiments without materially departing from the novel teachings and advantages of the present inventive concept. Accordingly, all such modifications are intended to be included within the scope of the present inventive concept as defined in the claims. In the claims, means-plus-function clauses are intended to cover the structures described herein as performing the recited function and not only structural equivalents but also equivalent structures. Therefore, it is to be understood that the foregoing is illustrative of various example embodiments and is not to be construed as limited to the specific example embodiments disclosed, and that modifications to the disclosed example embodiments, as well as other example embodiments, are intended to be included within the scope of the appended claims.

That which is claimed:

1. A cell fusion device comprising:
a chamber including a first input/output portion and a second input/output portion, the chamber configured to provide a space through which a fluid containing cells flows;
at least one cell fusion structure disposed within the chamber configured to form a fluidic channel through which the fluid flows, the at least one cell fusion structure including a capturing portion configured to capture the cell in the fluid and a fusion portion in fluid communication with the capturing portion, the fusion portion configured to provide a space for fusing at least two cells captured by the capturing portion and moved into the fusion portion in sequential order;
a deformable membrane structure disposed proximate the capturing portion of the cell fusion structure and configured to actuate to change a cross-sectional area of the fluidic channel of the capturing portion and to selectively capture the cell; and
a membrane control portion configured to apply a force to the deformable membrane structure.

2. The device of claim 1, wherein the cell fusion structure comprises at least first and second channel patterns formed on an inner wall defining the chamber, the first and second channel patterns configured to form the fluidic channel.

3. The device of claim 2, wherein the first and second channel patterns are arranged to face each other to form the capturing portion and the fusion portion.

4. The device of claim 2, wherein an inlet of the cell fusion structure has a first width, the capturing portion has a second width greater than the first width, and the fusion portion has a third width less than the second width.

5. The device of claim 1, wherein the deformable membrane structure comprises a gate membrane portion, the gate membrane portion configured to be deformed when a force is applied thereto so as to block the cell from passing through the capturing portion.

6. The device of claim 5, wherein the gate membrane portion is deformed by a force so as to close the inlet of the cell fusion structure.

7. The device of claim 5, wherein the membrane control portion comprises a membrane pressurizing portion adapted to apply the force to the gate membrane portion.

8. The device of claim 1, wherein the membrane control portion comprises a recess formed in an inner wall defining the chamber that extends across the capturing portion of the cell fusion structure.

9. The device of claim 8, wherein the deformable membrane structure comprises a deformable membrane to cover the recess.

10. The device of claim 1, wherein the membrane control portion is connected to a pneumatic supply source and configured to deform the deformable membrane structure.

11. The device of claim 1, further comprising a pair of electrode patterns disposed proximate opposing sides of the cell fusion structure and configured to provide an electrical signal to the cells disposed in the fusion portion.

12. The device of claim 11, wherein the electrode patterns are formed on an inner wall defining the chamber.

13. The device of claim 11, further comprising an electrical signal generator operably engaged with the electrode patterns so as to provide the electrical signal to the electrode patterns.

14. The device of claim 1, wherein a plurality of the cell fusion structures is arranged in a first direction to form one capturing array.

15. The device of claim 14, wherein a plurality of the capturing arrays is arranged in a second direction substantially perpendicular to the first direction.

16. A cell fusion method comprising:
providing a cell fusion device, the cell fusion device comprising:
a chamber including a first input/output portion and a second input/output portion, the chamber configured to provide a space through which a fluid containing cells flows;
at least one cell fusion structure disposed within the chamber configured to form a fluidic channel through which the fluid flows, the at least one cell fusion structure having a capturing portion and a fusion portion in fluid communication with the capturing portion; and
a deformable membrane structure disposed proximate the capturing portion of the cell fusion structure;
deforming the deformable membrane structure thereby changing a cross-sectional area of the fluidic channel of the capturing portion;
introducing a fluid containing cells into the chamber through the first input/output portion; and
fusing at least two cells disposed within the fusion portion of the cell fusion structure.

17. The method of claim 16, wherein deforming the deformable membrane structure comprises applying a pressure to the deformable membrane structure to block the cell entering through the capturing portion.

18. The method of claim 17, wherein blocking the cell from entering through the capturing portion comprises deforming the deformable membrane structure so as to close an inlet of the cell fusion structure.

19. The method of claim 16, wherein introducing the fluid containing cells into the chamber through the first input/output portion comprises:
introducing a fluid containing a first cell; and
introducing a fluid containing a second cell.

20. The method of claim 16, wherein fusing the at least two cells comprises applying an electrical signal to the cells disposed in the fusion portion.

21. The method of claim 16, further comprising culturing the fused cell in the chamber.

22. The method of claim 16, further comprising analyzing characteristics of the fused cell in the chamber.
